(19) United States
(12) Patent Application Publication
(10) Pub. No.: US 2008/0221585 A1
(43) Pub. Date: Sep. 11, 2008

(54) Lens Delivery System Cartridge

(76) Inventor: David A. Downer, Forth Worth, TX (US)

Correspondence Address: ALCON, IP LEGAL, TB4-8, 6201 SOUTH FREEWAY, FORT WORTH, TX, 76134, US

(21) Appl. No.: 11/684,337

(22) Filed: Mar. 9, 2007

(51) Int. Cl. A61F 9/00

(52) U.S. Cl. 606/107

(57) ABSTRACT

A cartridge for an IOL delivery system that folds open along its longitudinal axis on two pairs of hinges. The interior of the cartridge is hollow and contains a folding chamber connected to a nozzle. The folding chamber has a pair of rails that help to assist in providing an initial fold of the IOL and help prevent rotation of the IOL during injection.

This invention relates to intraocular lenses (IOLs) and more particularly to cartridges for use with devices use to inject IOLs into an eye.

### BACKGROUND OF THE INVENTION

The human eye in its simplest terms functions to provide vision by transmitting and refracting light through a clear outer portion called the cornea, and further focusing the image by way of the lens onto the retina at the back of the eye. The quality of the focused image depends on many factors including the size, shape and length of the eye, and the shape and transparency of the cornea and lens.

When trauma, age or disease cause the lens to become less transparent, vision deteriorates because of the diminished light which can be transmitted to the retina. This deficiency in the lens of the eye is medically known as a cataract. The treatment for this condition is surgical removal of the lens and implantation of an artificial lens or IOL.

While early IOLs were made from hard plastic, such as polymethylmethacrylate (PMMA), soft, foldable IOLs made from silicone, soft acrylics and hydrogels have become increasingly popular because of the ability to fold or roll these soft lenses and insert them through a smaller incision. Several methods of rolling or folding the lenses are used. One popular method is an injector cartridge that folds the lenses and provides a relatively small diameter lumen through which the lens may be pushed into the eye, usually by a soft tip plunger. The most commonly used injector cartridge design is illustrated in U.S. Pat. No. 4,681,102 (Bartell), and includes a split, longitudinally hinged cartridge. Similar designs are illustrated in U.S. Pat. Nos. 5,494,484 and 5,499,987 (Feingold) and U.S. Pat. Nos. 5,616,148 and 5,620,450 (Eagles, et al.). In an attempt to avoid the claims of U.S. Pat. No. 4,681,102, several solid cartridges have been investigated, see for example U.S. Pat. No. 5,275,604 (Rheinish, et al.) and U.S. Pat. No. 5,653,715 (Reich, et al.).

These prior art devices were intended to inject an IOL into the posterior chamber of an aphakic eye following removal of a cataractous lens. In such an eye, there is additional room to maneuver the IOL, such as turn the IOL over if the IOL is injected upside down. As a result, these prior art cartridges did not contain features to prevent the IOL from rotating within the cartridge.

With respect to IOLs intended to be planted into a phakic eye (an eye in which the natural lens remains), these lenses are either placed in the anterior chamber, or placed in the posterior chamber between the natural lens and the iris. Both styles of phakic lenses have a curvature or vault, requiring that the IOL be placed right side up. And in either case, the amount of room to maneuver the IOL is greatly restricted. Therefore, it is extremely important that the IOL be injected into the eye in the correct orientation.

Accordingly, a need continues to exist for an intraocular lens injection cartridge that helps prevent the lens from rotating during injection.

### BRIEF SUMMARY OF THE INVENTION

The present invention improves upon prior art by providing a cartridge for an IOL delivery system that folds open along its longitudinal axis on two pairs of hinges. The interior of the cartridge is hollow and contains a folding chamber connected to a nozzle. The folding chamber has a pair of rails that help to assist in providing an initial fold of the IOL and help prevent rotation of the IOL during injection.

It is accordingly an objective of the present invention to provide a cartridge for a lens delivery system that helps to prevent rotation of the lens during insertion.

It is a further objective of the present invention to provide a cartridge for a lens delivery system that has a folding chamber with a pair of rails.

It is a further an objective of the present invention to provide a cartridge for a lens delivery system that folds open along its longitudinal axis on two pairs of hinges.

Other objectives, features and advantages of the present invention will become apparent with reference to the drawings, and the following description of the drawings and claims.

### BRIEF DESCRIPTION OF THE DRAWINGS

FIG. 1 is an enlarged top perspective view of the lens delivery system cartridge of the present invention.

FIG. 2 is an enlarged bottom perspective view of the lens delivery system cartridge of the present invention.

FIG. 3 is an enlarged side elevational view of the lens delivery system cartridge of the present invention.

FIG. 4 is an enlarged proximal end view of the lens delivery system cartridge of the present invention shown in the closed position and containing a folding IOL.

### DETAILED DESCRIPTION OF THE PREFERRED EMBODIMENTS

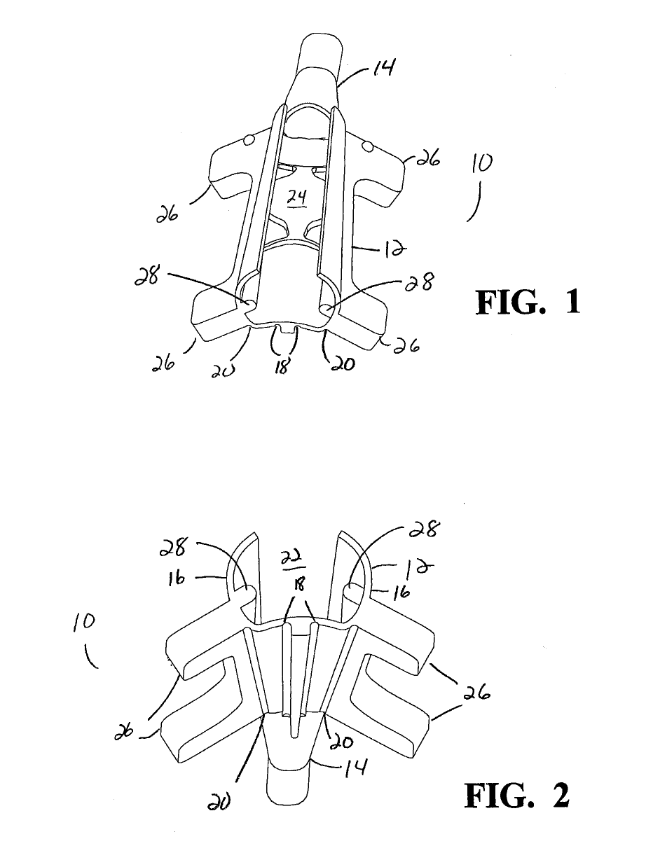

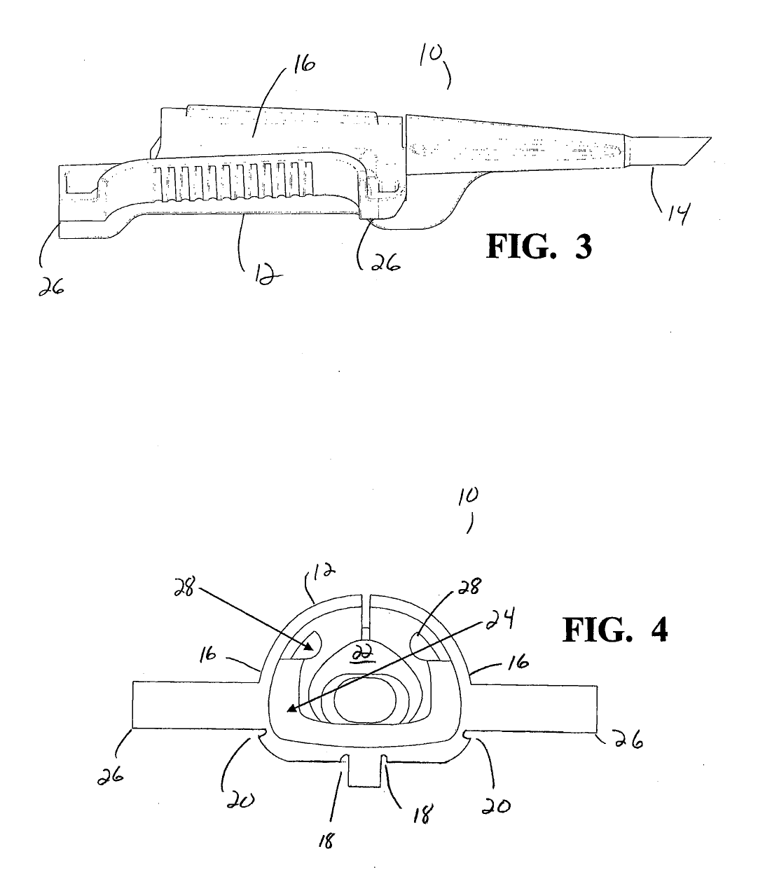

As best seen in FIGS. 1-3, lens cartridge 10 of the present invention generally includes body 12 and nozzle 14. Cartridge 10 can be molded from any suitable thermoplastic, such as polypropylene, and the thermoplastic may contain a lubricity enhancing agent such as those disclosed in U.S. Pat. No. 5,716,364, the entire contents of which are incorporated herein by reference. Nozzle 14 may be integrally formed with body 12. Nozzle 14 preferably is tubular, round, oval or elliptical in cross-section and has a cross-sectional area of between around 1.0 mm² to around 2.6 mm². Body 12 is formed by two hemispherical halves 16 connected by hinge pairs 18 and 20. When collapsed or folded together, halves 16 form body 12 having a hollow interior folding chamber 22 into which IOL 24 may be placed. This folding may be assisted by use of finger grips 26. Projecting into interior 22 of body 12 is a pair of rails 28.

In use, IOL 24 is placed within chamber 22 of body 12 below rails 28. Halves 16 are collapsed together, initially by flexing of hinge pairs 18, which impart an initial fold on IOL 24 that ensures that IOL 24 bends or flexes downwardly toward hinge pair 18. Rails 28 prevent IOL 24 from rotating within body 12 during folding. As halves 16 continue to be collapsed together, further folding of IOL 24 is accomplished by flexing of hinge pairs 20, as best seen in FIG. 4

While certain embodiments of the present invention have been described above, these descriptions are given for purposes of illustration and explanation. Variations, changes, modifications and departures from the systems and methods disclosed above may be adopted without departure from the scope or spirit of the present invention.

I claim:

1. An intraocular lens delivery system cartridge, comprising:
a) a body, the body formed by a pair of body halves connected by two pairs of hinges, the body halves defining a hollow interior chamber in the body; and
b) a tubular nozzle connected to the body.

2. An intraocular lens delivery system cartridge, comprising:
a) a body, the body having a hollow interior chamber;
b) a tubular nozzle connected to the body; and
c) a pair of rails projecting into the hollow interior chamber in the body.

3. An intraocular lens delivery system cartridge, comprising:
a) a body, the body formed by a pair of body halves connected by two pairs of hinges, the body halves defining a hollow interior chamber in the body;
b) a tubular nozzle connected to the body; and
c) a pair of rails projecting from the body halves and into the hollow interior chamber in the body.
